(19) United States
(12) Patent Application Publication
(10) Pub. No.: US 2022/0134055 A1
(43) Pub. Date: May 5, 2022

(54) CAPTIVATION CATHETERS AND RELATED METHODS

(71) Applicant: Teleflex Life Sciences Limited, Valletta (MT)

(72) Inventors: Jordan Berg, Brooklyn Park, MN (US); Andrew Blommer, Plymouth, MN (US); Jack Fischbach, Minneapolis, MN (US)

(73) Assignee: Teleflex Life Sciences Limited, Valletta (MT)

(21) Appl. No.: 17/412,316

(22) Filed: Aug. 26, 2021

Related U.S. Application Data
(60) Provisional application No. 63/109,106, filed on Nov. 3, 2020.

(51) Int. Cl. A61M 25/01; A61M 25/09; A61M 25/00

(52) U.S. Cl. CPC A61M 25/0138; A61M 25/09; A61M 25/0053; A61M 2205/584; A61M 2025/09008; A61M 2025/0008; A61M 25/0052

(57) ABSTRACT

A captivation catheter for use with a guide catheter and a guidewire or a treatment catheter. The captivation catheter includes a relatively rigid elongate member and a captivation mechanism such as an inflatable balloon capable of fixing the guidewire or the treatment catheter in place against an inner surface of the guide catheter. The captivation catheter can include a clip with an open end and a closed end that is placed on the captivation catheter at markings present for compatibility with a particular working length of the guide catheter. The markings can be visible at different viewing orientations.

This non-provisional patent document claims the benefit of priority under 35 U.S.C. § 119(e) to U.S. Provisional Patent Application Ser. No. 63/109,106, entitled “CAPTIVATION CATHETERS AND RELATED METHODS” and filed Nov. 3, 2020, which is herein incorporated by reference in its entirety.

### TECHNICAL FIELD

This patent document relates to medical devices. More particularly, but not by way of limitation, the patent document relates to percutaneous devices and methods suitable for use in minimally invasive treatment of various disorders and diseases.

### BACKGROUND

Minimally invasive medicine and the practice of gaining access into a blood vessel or other hollow bodily structure to facilitate the subsequent introduction and placement of catheters or other interventional medical devices has been evolving since creation of the Seldinger technique in the early 1950s.

An important advance during this evolution was gaining the ability to exchange catheters or other interventional medical devices over a single indwelling guidewire without displacing the guidewire proximally, which surrenders access to a desired diseased site, or distally, which risks perforation or other guidewire-induced patient injury. This “over-the-wire” (OTW) or “long wire” exchange technique requires a long guidewire length so that the guidewire can be handled and stabilized from outside a patient's body at all times during a procedure. The portion of the guidewire extending out of the patient must be at least slightly longer than the full-length lumen of the catheter (or other interventional medical device) to be employed. In this way, a proximal end of the guidewire can protrude from a proximal end of the catheter and can be held by an operating physician or his/her assistant to maintain the guidewire's indwelling positioning.

To exchange one catheter for another using the OTW technique, the physician and assistant must make a series of well-coordinated, one-to-one movements between the guidewire and each catheter. The assistant pushes the guidewire the same amount as the physician pulls back on a first catheter until the first catheter is completely outside of the patient and the physician gains control over the guidewire at its entry site into the patient. The assistant then pulls the first catheter off the guidewire and backloads a second catheter over the guidewire and into the patient to perform a second operation, requiring this same push-pull technique in reverse. Once the second catheter is fully loaded onto the guidewire, the guidewire's proximal end can protrude from the catheter's proximal end and can be held by the assistant, typically standing well apart from the physician. These exchange maneuvers must be guided fluoroscopically to monitor distal guidewire position, thereby increasing the dose of radiation to which the patient, physician and assistant are exposed during the procedure. Moreover, these exchange maneuvers are prone to error resulting in loss of indwelling guidewire position.

A technique that allows for a much shorter guidewire length to be used and more physician control over the guidewire was developed to simplify catheter exchanges. Known as the “rapid exchange,” “monorail,” or “short wire” technique, it is used in conjunction with rapid exchange, monorail or short wire catheters, which include a shortened guidewire passageway extending along only a portion of a catheter's length. The rapid exchange technique differs from the OTW technique in that a guidewire is fed into the shortened passageway and exits at a point between the catheter's distal and proximal ends via a port or channel formed in the side of the catheter. The physician can perform a short wire catheter exchange by handling a guidewire portion length slightly longer than a length of the shortened passageway (instead of a guidewire length slightly longer than an entire catheter, as is the case when using the OTW technique). This facilitates the physician maintaining control of the guidewire at all times and reduces the need for coordinating x-ray guided push-pull exchange movements with an assistant.

As vascular interventions grow in complexity, there are an increasing number of special-purpose OTW catheters embodying full-length guidewire lumens; however, physicians generally prefer the convenience of using a short rapid exchange length guidewire versus the more cumbersome OTW length guidewire.

When OTW catheters are used in combination with rapid exchange length guidewires, an operating physician is unable to maintain a hold onto the guidewire during an entire catheter exchange process. When the guidewire can no longer be held, guidewire position can easily be lost if the OTW catheter pulls the guidewire back with it while being withdrawn. In a similarly undesirable manner, when trying to advance the OTW catheter over the rapid exchange guidewire, the guidewire can be unintentionally pushed distally by the catheter during the time when the guidewire's proximal end cannot be held and stabilized by the physician or his/her assistant. This advancement of the guidewire poses a potential risk of vessel perforation or other damage, because the guidewire's distal tip can advance into a small or diseased vessel that the physician does not intend to cannulate. Alternatively, redundant loops of guidewire can form in front of the OTW catheter being advanced leading to loss of wire control and positioning. Any of these uncontrolled guidewire movements arising from OTW catheter movements over rapid exchange length guidewires can lead to procedural inefficiency, procedural failure and/or patient complications.

### OVERVIEW

Devices and methods to fix indwelling guidewire positioning during percutaneous procedures are needed, particularly when the use of an OTW catheter and a rapid exchange length guidewire are anticipated.

A captivation catheter for use with a guide catheter and a guidewire, particularly a rapid exchange length guidewire, can include a relatively rigid elongate member and a captivation mechanism such as an inflatable balloon capable of fixing the guidewire in place against an inner surface of the guide catheter. The elongate member can have a tubular shape defining a lumen sized and shaped to communicate an inflation fluid. The elongate member can have an outer diameter smaller than a lumen of the guide catheter. The inflatable balloon is of a size and shape to engage or lock the guidewire against an inner surface of the guide catheter when inflated. This engagement or locking can facilitate the exchange of a treatment catheter while maintaining the position of the guidewire within the desired target vessel or other hollow structure. The captivation catheter can include a clip with an open end and a closed end that is placed on the captivation catheter at markings present for compatibility with a particular working length of the guide catheter. Different markings can be visible at different viewing orientations.

The present devices and methods include means to engage an intermediate or distal end portion of a guidewire against an inner surface of a guide catheter. Once the guidewire position is locked by its engagement against the guide catheter's inner surface, withdrawal of a first OTW catheter and subsequent introduction of a second OTW catheter over the guidewire is possible without moving the guidewire longitudinally relative to the guide catheter.

Aspects of the present invention include a captivation catheter for use with a guide catheter and a guidewire or a treatment catheter. The captivation catheter includes an elongate member extending from a proximal hub to a distal end, and a distal portion of the elongate member can include a helical cut extending through a wall of the elongate member. The captivation catheter includes a captivation balloon positioned over the helical cut, the captivation balloon configured for expansion within the guide catheter to secure the guidewire or the treatment catheter between an outer surface of the captivation balloon and an inner surface of the guide catheter. The helical cut allows for inflation of the captivation balloon and increased shaft flexibility relative to a proximal portion of the elongate member.

In some aspects, the pitch of the helical cut decreases in a proximal-to-distal direction along the distal portion of the elongate member. A through hole having a radius greater than the width of the helical cut may be found at each end of the helical cut to inhibit crack propagation from the end of the cut. In some aspects, an outer diameter of the elongate member decreases in a proximal-to-distal direction along the elongate member.

In some aspects, the elongate member includes at least first and second markings indicative of a position of the captivation catheter relative to the guide catheter, the first marking visible when the elongate member is at a first orientation but not a second orientation in a surgical field and the second marking visible when the elongate member is at the second orientation but not the first orientation in the surgical field. The first marking can include a color marking and the second marking can include a surface texture marking. In some aspects, the first and second markings are positioned adjacent to each other.

In some aspects, the captivation catheter includes a clip having an open first end that is attachable to the captivation catheter, and a closed second end that is opposite the first end. The clip is configured to be removed from the elongate member by depressing or pinching portions of the clip adjacent the first end.

In some aspects, a center of the captivation balloon post-expansion is offset relative to an axis of the elongate member.

These illustrative aspects are mentioned not to limit or define the disclosure, but to provide examples to aid understanding thereof. Additional aspects are discussed throughout the description in this document.

### BRIEF DESCRIPTION OF THE DRAWINGS

Features, aspects, and advantages of the present disclosure are better understood when the following Detailed Description is read with reference to the accompanying drawings. In the drawings, like numerals can be used to describe similar features and components throughout several drawings. The drawings illustrate generally, by way of example, but not by way of limitation, various device and method aspects discussed in this patent document.

FIG. 1 illustrates a plan view of a guide catheter advanced through an aorta to an ostium of a coronary artery, where the aorta and coronary artery are illustrated in a cross-sectional view, a guidewire, and a treatment catheter.

FIG. 2 illustrates a cross-sectional view of a guide catheter, a guidewire, a treatment catheter, and a captivation catheter, all present in an aorta according to one aspect of the present invention.

FIG. 3A illustrates a plan view of a captivation catheter according to one aspect of the present invention.

FIG. 3B illustrates an enlarged plan view of area A from FIG. 3A of a captivation catheter according to one aspect of the present invention.

FIG. 4A illustrates a plan view of an elongate member of a captivation catheter according to one aspect of the present invention.

FIG. 4B illustrates an enlarged plan view of area A from FIG. 4A of an elongate member of a captivation catheter according to one aspect of the present invention.

FIG. 5A illustrates a plan view of an elongate member of a captivation catheter according to one aspect of the present invention.

FIG. 5B illustrates a cross section view of line B from FIG. 5A of an elongate member of a captivation catheter according to one aspect of the present invention.

FIG. 5C illustrates an enlarged plan view of area C from FIG. 5A of an elongate member of a captivation catheter according to one aspect of the present invention.

FIG. 6 illustrates a side view of a distal section of a captivation catheter according to one aspect of the present invention.

FIG. 7A illustrates an isometric view of a clip according to one aspect of the present invention.

FIG. 7B illustrates an end view of a clip according to one aspect of the present invention.

FIG. 7C illustrates a side view of a clip according to one aspect of the present invention.

FIG. 8 illustrates a plan view of a captivation catheter including a clip according to one aspect of the present invention.

### DETAILED DESCRIPTION

Numerous specific details are set forth herein to provide a thorough understanding of the claimed subject matter. However, those skilled in the art will understand that the claimed subject matter may be practiced without these specific details. In other instances, methods, devices, or assemblies that would be known by one of ordinary skill have not been described in detail so as not to obscure claimed subject matter.

The use of “adapted to” or “configured to” herein is meant as open and inclusive language that does not foreclose devices adapted to or configured to perform additional tasks or steps. The use of “proximal” and “distal” herein refers to relative positions with respect to a user of the elongate, minimally invasive devices, where “proximal” means relatively towards the user and “distal” means relatively away from the user. Headings, lists, and numbering included herein are for ease of explanation only and are not meant to be limiting. All numeric values are assumed to be modified by the term “about,” whether or not explicitly indicated. The term “about” generally refers to a range of numbers that one of ordinary skill would consider equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.).

As discussed above, existing techniques used to fix the position of indwelling guidewire positioning during percutaneous procedures are insufficient. A number of minimally invasive intravascular procedures have been developed. Some of these procedures include balloon angioplasty and/or the delivery of intravascular stents. At least some of these procedures utilize a guide catheter to help “guide” a therapeutic device to a target. The use of a guide catheter may include advancing the guide catheter through a body lumen (e.g., blood vessel) to a position near a target, advancing a guidewire through the guide catheter, and advancing the therapeutic catheter over the guidewire toward the target. In some instances, it may be desirable to remove the therapeutic catheter and then advance a different therapeutic catheter toward the target. For a number of reasons it may be desirable to maintain the position of the guidewire (which may be desirably positioned relative to the target) during the catheter exchange.

Oftentimes, after a catheter or other interventional medical device is inserted into a patient, it can be necessary to withdraw the catheter or device to substitute an alternate-sized catheter or device. For example, the profile of a deflated balloon of a dilatation catheter can sometimes be too large to fit through a diseased site (e.g., a stenosis) to be treated, or the balloon profile may be so small that, upon inflation of the balloon, the diseased site is not sufficiently dilated. When this occurs, the dilatation balloon catheter needs to be exchanged for one of a different (smaller or larger) size, so that the diseased site can be crossed and properly treated upon inflation of the balloon. The catheter or device may also or alternatively have poor control or low flexibility resulting in an inability to track to an anatomic landmark distal to the diseased site. In this case, the catheter or device must be exchanged for one with better tracking characteristics so that the anatomic landmark can be reached. These substitutions are completed during a catheter or device exchange.

The present percutaneous devices and methods allow for reliable insertion and removal of both OTW and rapid exchange catheters or other interventional medical devices over a guidewire of any length (including rapid exchange guidewires having a length of about 190 centimeters (cm) or less), while maintaining the position of the guidewire relative to a guide catheter or an anatomic landmark within vasculature. Maintaining the indwelling position of the guidewire during such interventional medical device insertions and removals reduces the need for coordinating x-ray guided push-pull exchange movements as would otherwise be required.

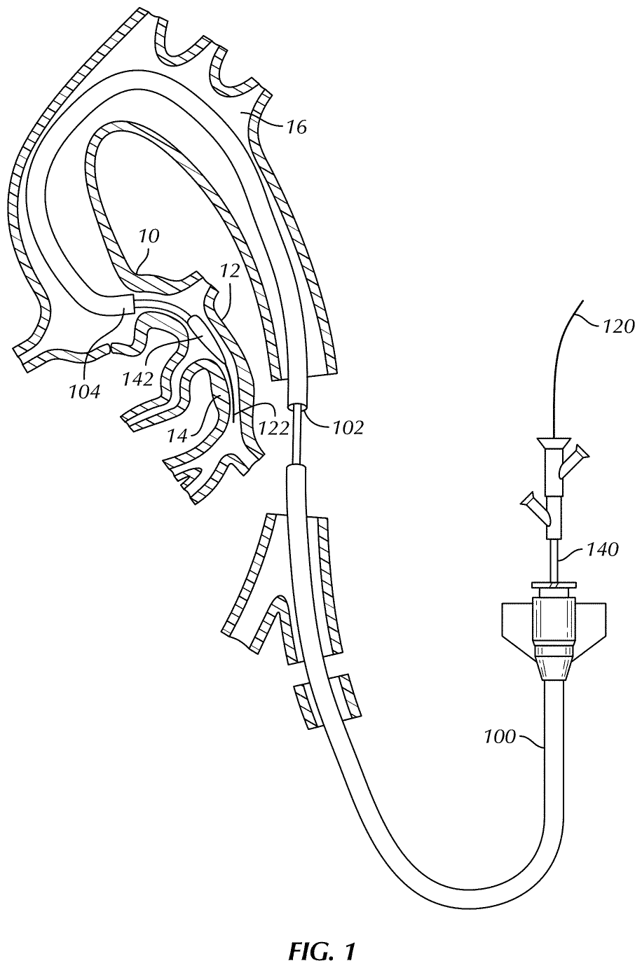

FIG. 1 illustrates a plan view of a guide catheter advanced through an aorta to an ostium of a coronary artery, where the aorta and coronary artery are illustrated in a cross-sectional view, a guidewire, and a treatment catheter. Minimally invasive cardiac interventions, such as percutaneous transluminal coronary angioplasty procedures, are utilized throughout the world and typically include the use of a guide catheter 100, as illustrated in FIG. 1. The guide catheter 100 is an long tubular member having a guide catheter lumen 102 throughout its length. The guide catheter 100 can be formed of polyurethane, for example, and can be shaped to facilitate its passage to a coronary ostium 10 or other region of interest within a patient's body. In the example of FIG. 1, a 6 French (F), 7 F or 8 F guide catheter 100 can be inserted at a femoral artery and advanced through the aorta 16 to a position adjacent to the ostium 10 of a coronary artery 12. The diameter and rigidity of the guide catheter 100 oftentimes does not permit it to be advanced beyond the ostium 10 into the coronary artery 12 requiring treatment, and thus, a treatment catheter 140 must be advanced independently of the guide catheter 100 to reach a treatment site 14.

Maintaining the position of the guide catheter distal portion 104 at the ostium 10 can be desirable to facilitate the treatment portion 142 of the treatment catheter 140 successfully reaching the treatment site 14. When resistance is encountered as attempts are made to deliver the treatment catheter 140, the guide catheter 100 can back-out or withdraw from the ostium 10. A heart's intrinsic beat can also cause the guide catheter distal portion 104 to lose its positioning or otherwise be shifted so that it no longer is positioned to guide the treatment catheter 140 to the treatment site 14. Because of this shift away from the ostium 10, access to the coronary artery 12 and the treatment site 14 can require repeated repositioning of the guide catheter 100 in order to bring the guide catheter distal portion 104 back into engagement with the ostium 10.

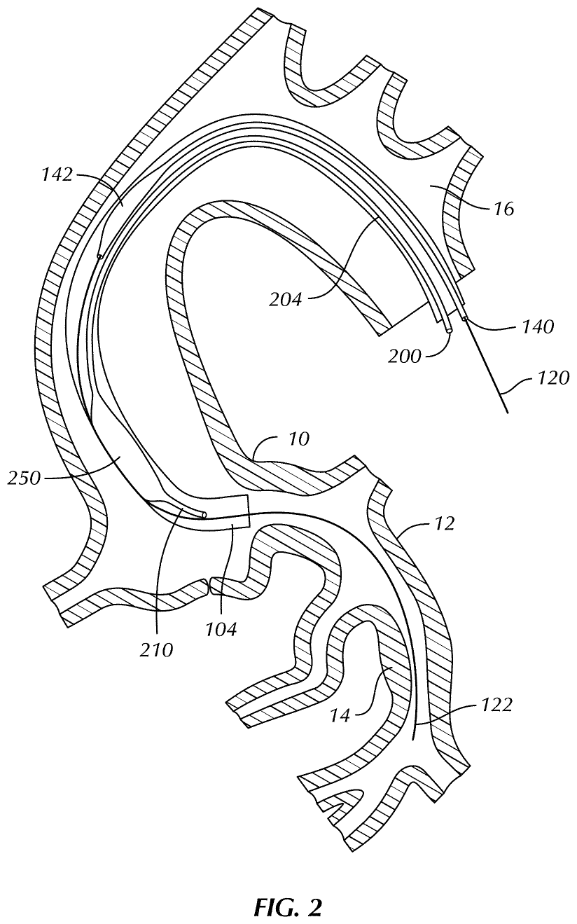

FIG. 2 illustrates a cross-sectional view of a guide catheter, a guidewire, a treatment catheter, and a captivation catheter, all present in an aorta according to one aspect of the present invention. A captivation catheter 200 can improve access to a coronary artery 12 and a treatment site 14. The captivation catheter 200 includes a relatively flexible elongate member 204 and an inflatable portion 250 (e.g., a captivation balloon illustrated in FIG. 2). In one aspect of a method of use of a captivation catheter, the captivation catheter 200 is advanced to an area proximal to the guide catheter distal portion 104 after the guidewire distal portion 122 has been advanced to a desired location near a treatment site 14. A treatment catheter 140 is advanced over the guidewire 120 to the treatment site 14, where treatment can be performed. During this step, the inflatable portion 250 of the captivation catheter 200 is in a configuration that allows the treatment portion 142 to pass and continue distally to the treatment site 14.

Still referring to FIG. 2, after the treatment portion 142 has been used at the treatment site 14, it may be desirable to exchange the treatment catheter 140 for a different treatment catheter. In this case, the treatment portion 142 is withdrawn along the guidewire 120 proximally to a position proximal of the inflatable portion 250. The inflatable portion 250 can then be engaged to capture (or fix the position of) the guidewire 120. In the aspect illustrated in FIG. 2, the inflatable portion 250 is an expandable balloon that fixes the position of the guidewire 120 against the inner surface of the guide catheter lumen 102. By fixing the position of the guidewire 120 relative to the guide catheter 100, the position of the guidewire distal portion 122 relative to the treatment site 14 can be maintained while the treatment catheter 140 is withdrawn over the guidewire 120 and out of the guide catheter 100.

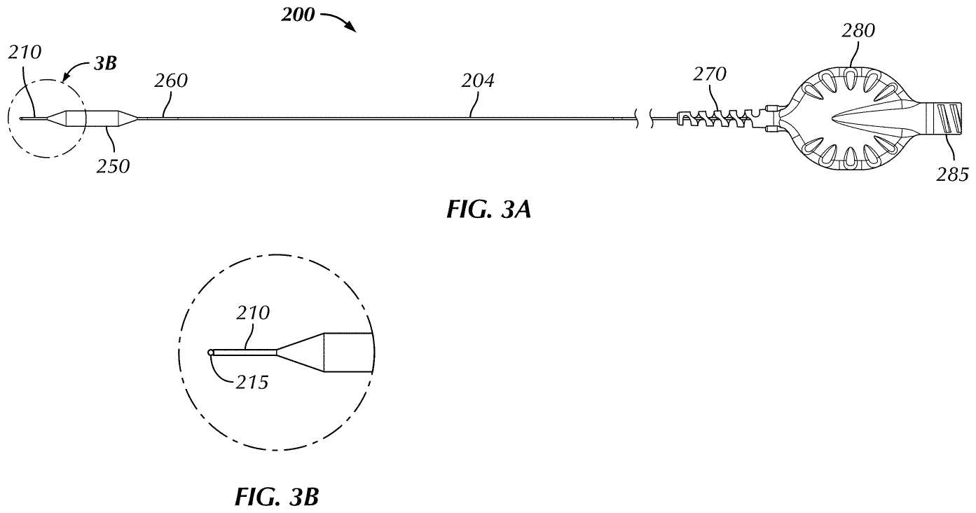

FIG. 3A illustrates a plan view of a captivation catheter and FIG. 3B illustrates an enlarged plan view of area A from FIG. 3A of a captivation catheter. The captivation catheter 200 includes an elongate member 204 in fluid communication with a hub 280, which includes a fluid fitting 285. The elongate member 204 can be connected with the hub 280 via a strain relief section 270. The distal end region of the captivation catheter 200 includes a distal wire tip 215, which can be configured with a rounded end to facilitate movement of the captivation catheter 200 through a guide catheter.

The inflatable portion 250 of the captivation catheter can be attached to the elongate member 204 at the captivation catheter distal portion 210 and at an inflatable portion proximal section 260 and can be configured to be concentric with the elongate member 204. The inflatable portion 250 can include one or multiple polymer layers. In an example, the inflatable portion 250 includes a single polymer layer formed of nylon, polyether block amides, polyethylene terephthalate (PET), or polyurethane. In another example, the inflatable portion 250 includes an inner polymer layer and an outer polymer layer; the inner polymer layer can include a high durometer polymer to increase resistance to bursting and provide enhanced outward force, and the outer polymer layer can include a lower durometer polymer providing flexibility and conformance with a vessel wall. A radiopaque marker band may be located in the approximate longitudinal midway point of the inflatable portion to facilitate positioning using fluoroscopy.

Other examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

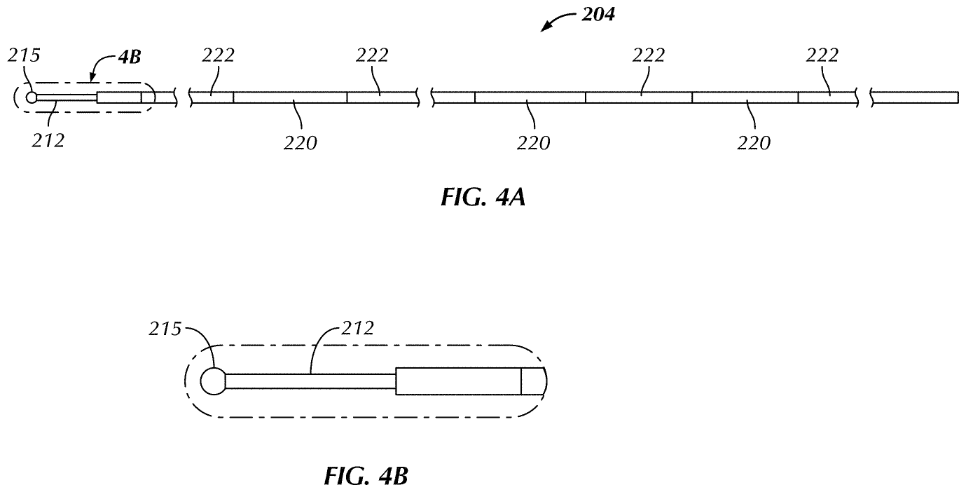

FIG. 4A illustrates a plan view of an elongate member of a captivation catheter and FIG. 4B illustrates an enlarged plan view of area A from FIG. 4A of an elongate member of a captivation catheter. The elongate member 204 is preferably formed of a material and/or of a configuration with relatively high column strength or “pushability.” For example, a metal hypotube, which is commonly known in the relevant art as a long tube often including micro-engineered features along its length, is a material configuration particularly suitable for use as the elongate member 204. Some other examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material. A specific example of a suitable material and configuration for the elongate member 204 is a 304 stainless steel hypotube.

Referring still to FIG. 4A and FIG. 4B, the elongate member 204 can include a distal wire 212 that is attached to the distal end region of the elongate member 204. The distal wire 212 includes a rounded distal wire tip 215. Preferably, the distal wire 212 and the distal wire tip 215 are formed from a radiopaque material. Radiopaque materials are understood to be materials capable of producing a relatively bright image on a fluoroscopy screen or another imaging technique during a medical procedure. This relatively bright image aids the user of the captivation catheter 200 in determining its location with the anatomy. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the captivation catheter 200 to achieve a similar result. A specific example of a suitable material for the distal wire 212 and the distal wire tip 215 is a platinum/iridium wire having a 90:10 platinum:iridium content.

FIG. 4A illustrates first marked regions 220 on the elongate member 204. The first marked regions 220 are regions that have been processed to produce surface markings or surface texture. The first marked regions 220 can include color marking applied to the region, a surface texture marking applied to the region, or a combination of a color marking and a surface texture marking. A surface texture can be applied such that the marking is more prominently visible when viewed from a particular viewing angle and less visible (or not visible) when viewed from other viewing angles. The first marked regions 220 indicate a linear position of the captivation catheter relative to the guide catheter.

FIG. 4A also illustrates second marked regions 222 on the elongate member 204. Similar to the first marked regions 220, the second marked regions 222 are regions that have been processed to produce surface markings or surface texture. The second marked regions 222 can include color marking applied to the region, a surface texture marking applied to the region, or a combination of a color marking and a surface texture marking. A surface texture can be applied such that the marking is more prominently visible when viewed from a particular viewing angle and less visible (or not visible) when viewed from other viewing angles. The marked regions can be formed to have a minimum Stouffer scale darkness value of 5, although other values may also be useful.

FIG. 4A illustrates that the first marked regions 220 and the second marked regions 222 are positioned adjacent to each other. A user can determine the linear position of the captivation catheter relative to the guide catheter from various angles of view in the surgical field because of the variable visual prominence of the first marked regions 220 and the second marked regions 222. For example, a first marked region 220 may be visible when the elongate member is at a first orientation but not a second orientation in a surgical field and the second marked region 222 may be visible when the elongate member is at the second orientation but not the first orientation in the surgical field. As discussed in further detail herein, the marked regions are used for placement of a clip. The first marked regions 220 and the second marked regions 222 are positioned adjacent to each other and at any given time, the physician may only see one of the color marking or the texture marking based on the viewing angle. That is, the first marked regions 220 and second marked regions 222 are configured such that one region is always visible regardless of the viewing angle. FIG. 4A depicts multiple pairs of first and second marked regions, which allows for use of the captivation catheter with guide catheters of varying lengths, as is discussed in further detail herein. Optionally, each of the multiple pairs of first and second marked regions can have a distinguishing characteristic to allow the pairs to be distinguishable from one another.

Referring still to FIG. 4A, the overall length of the elongate member 204 can be from about 110 cm to about 160 cm and more preferably about 140 cm. The distal wire 212, including the distal wire tip 215, can be from about 0.2 cm to about 0.6 cm and more preferably about 0.3 cm. The length of the elongate member 204 from the distal wire tip to the distal most first marked region 220 can be about 97 cm. The length of each of the first marked regions 222 and the second marked regions 222 can be from about 0.1 cm to about 1.0 cm and more preferably about 0.5 cm. The length of the elongate member 204 from the distal wire tip to the next distal most first marked region 220 can be about 107 cm.

The most distal marked region 220 and the next most distal marked region 220 are positioned on the elongate member 204 such that these marked regions 220 are useful to identifying when the distal wire tip 215 of the elongate member 204 is near the distal end of a guide catheter. Conventional guide catheters can be 90 cm long or 100 cm long. The location of a first marked region 220 at about 97 cm, and second marked region 222 proximal of the first marked region 220, can be useful for identifying that the distal wire tip 215 of the elongate member 204 is near the distal end of a 90 cm guide catheter. The location of a first marked region 220 at about 107 cm, and second marked region 222 proximal of the first marked region 220, can be useful for identifying that the distal wire tip 215 of the elongate member 204 is near the distal end of a 100 cm guide catheter.

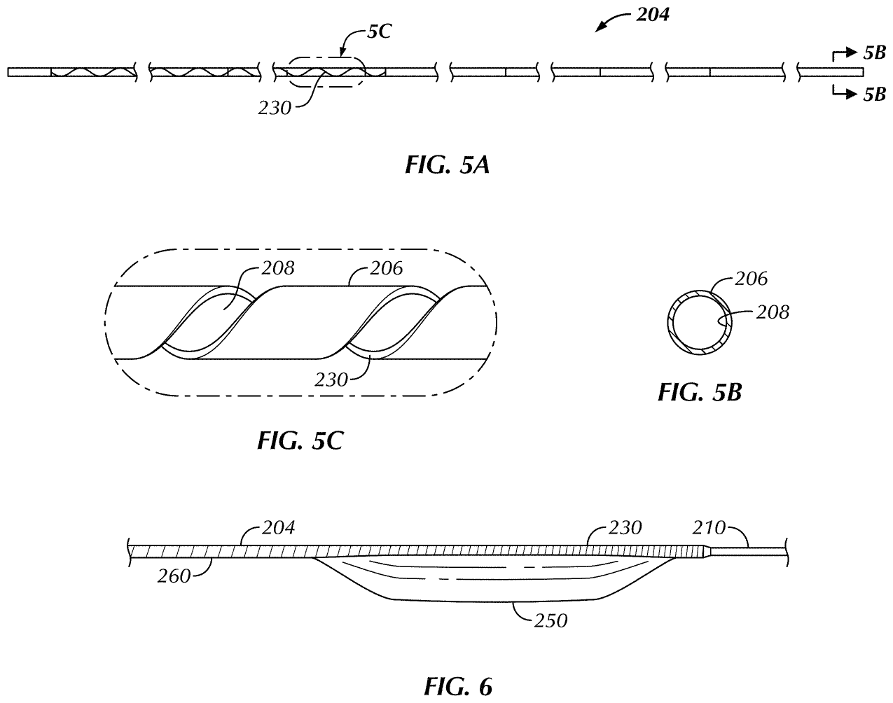

FIG. 5A illustrates a plan view of an elongate member of a captivation catheter, FIG. 5B illustrates a cross section view of line B from FIG. 5A, and FIG. 5C illustrates an enlarged plan view of area C from FIG. 5A. FIG. 5A illustrates a helical cut 230 present on the elongate member 204 in a region towards the distal end of the elongate member 204. The helical cut 230 may be counterclockwise when viewed proximal-to-distal or it may be clockwise when viewed proximal-to-distal.

The helical cut 230 can begin from about 0.1 cm to about 1.0 cm from the distal end of the elongate member 204 and more preferably begins about 0.2 cm from the distal end of the elongate member 204. The helical cut 230 can have a length of from about 8.0 cm to about 12.0 cm and more preferably a length of about 10.0 cm. FIG. 3C illustrates that the helical cut 230 extends through the wall of the elongate member 204. The wall of the elongate tube 204 is illustrated in FIG. 5B as the area between the outer surface 206 and the inner surface 208 of the elongate member 204. The wall of the elongate tube 204 may have a thickness of from about 0.01 cm to about 0.03 cm and more preferably about 0.02 cm. The width of the helical cut 230 may be from about 0.001 cm to about 0.005 cm and more preferably about 0.0025 cm.

In certain aspects, the pitch of the helical cut 230 decreases in a proximal-to-distal direction along the elongate member 204. For example, the pitch of the helical cut 230 may be at least about 0.25 cm at its proximal end and reduce to no more than about 0.013 cm at its distal end. More preferably, the pitch of the helical cut 230 may be at least about 0.23 cm at its proximal end and reduce to no more than 0.038 cm at its distal end. The pitch of the helical cut 230 may reduce at a continuous linear rate, a continuous geometric rate, a discontinuous rate, or combinations of such rates.

In certain aspects, the distal portion of the helical cut 230 terminates in a through hole having a radius greater than the width of the helical cut 230 to inhibit crack propagation from the end of the cut. In certain aspects, the proximal portion of the helical cut 230 terminates in a through hole having a radius greater than the width of the helical cut 230 to inhibit crack propagation.

In certain aspects, the outer diameter of the elongate member 204 decreases in a proximal-to-distal direction along the elongate member 204. That is, the elongate member 204 includes a tapered section. The tapered section may reduce at a continuous linear rate, a continuous geometric rate, a discontinuous rate, or combinations of such rates. The diameter at the proximal end of the tapered section may be from at least about 0.046 cm and may taper to at least about 0.039 cm at the distal end of the tapered section.

FIG. 6 illustrates a side view of a distal section of a captivation catheter according to one aspect of the present invention. The entire helical cut 230 on the elongate member 204 is illustrated as being encapsulated by the inflatable portion 250, beginning proximally from an area on the elongate member 204 where the inflatable portion proximal section 260 is attached and ending distally at an area on the captivation catheter distal portion 210. The inflatable portion 250 is attached to the elongate member 204 in these two locations such that a fluid-tight seal is formed. This fluid-tight seal makes it possible to inflate the inflatable portion 250 by filling the inflatable portion 250 with fluid transmitted through the helical cut 230 on the elongate member 204. Thus, the helical cut 230 functions both as a conduit for inflation fluid for the inflatable portion 250 and as a means to impart flexibility to the distal portion of the captivation catheter. By varying the pitch of the helical cut 230, it is possible to vary the flexibility of the distal portion of the captivation catheter.

FIG. 6 illustrates that the inflatable portion 250 can be arranged asymmetrically on the elongate member 204. That is, the inflatable portion 250 inflates preferentially in one radial direction away from the longitudinal axis of the elongate member 204 rather than uniformly about the longitudinal axis of the elongate member 204. When the elongate member 204 is positioned against one side of the inner surface of the lumen of a guide catheter, the asymmetric shape of the inflatable portion 250 can be advantageous for capturing a guidewire against the opposite inner surface of the lumen of the guide catheter by increasing the capturing surface area of the inflatable portion 250 on one side of the captivation catheter. A larger capturing surface area can facilitate initial capture of the guidewire and ensure stable captivation while devices are withdrawn proximally along the guidewire.

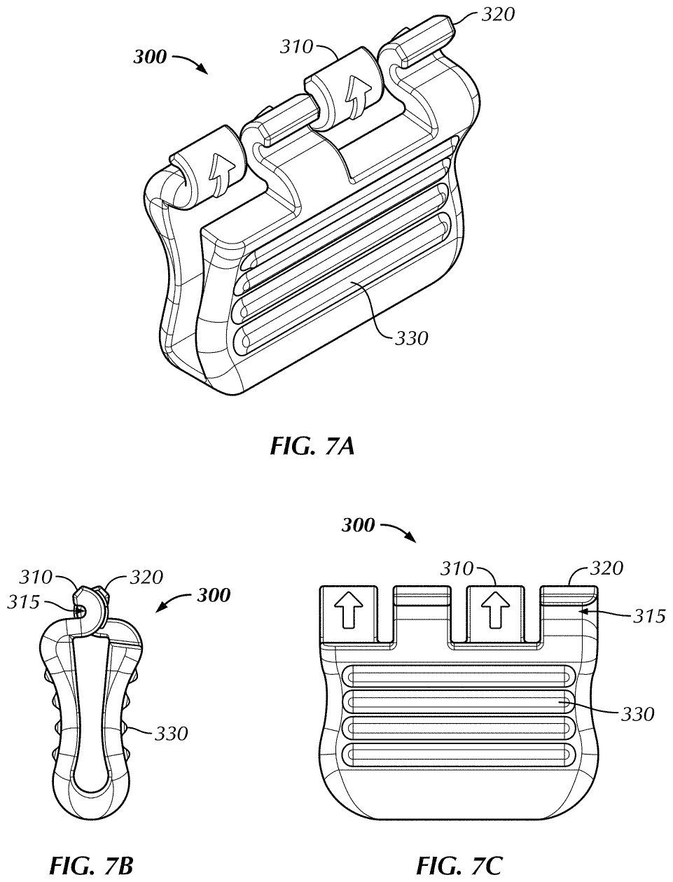

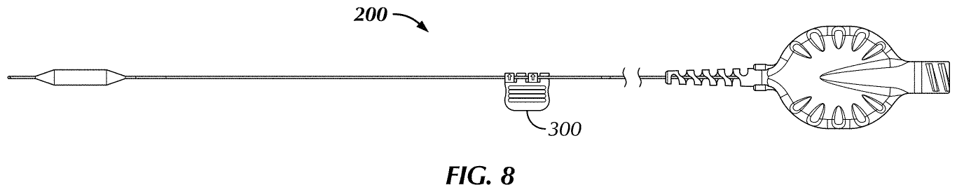

FIG. 7A illustrates an isometric view of a clip. FIG. 7B illustrates an end view of the clip, and FIG. 7C illustrates a side view of the clip according to one aspect of the present invention. The clip 300 can include an open end and a closed end, with the open end including at least a first arm 310 and a second arm 320. Each of the first arm 310 and second arm 320 may include a curved shape with a groove 315 where the first arm 310 and the second arm 320 are arranged such that the groove 315 on each faces away from the other. The clip 300 can include a grip section 330 that allows a user to pinch the clip together such that the first arm 310 and the second arm 320 move away from each other. When the clip 300 is pinched, the elongate member of a captivation catheter can be placed in the area now opened between the first arm 310 and the second arm 320. When the user stops pinching the grip sections 330 of the clip 300, the clip 300 is allowed to return to its original position and the elongate member fits into each of the grooves 315 on the first arm 310 and the second arm 320. Thus, the clip 300 is now releasably attached to the captivation catheter. FIG. 8 illustrates an arrangement where the clip 300 is releasably attached to the captivation catheter 200. The user can pinch the grip sections 330 of the clip 300 together to remove the clip 300 from the captivation catheter 200.

The clip 300 illustrated in FIGS. 7A, 7B, 7C, and 8 contains 2 first arms and 2 second arms. Generally, clips should include at least one first arm and one second arm and can contain multiple first arms and multiple second arms. Clips need not include the same number of first and second arms.

The aspects disclosed herein illustrate a captivation catheter that allows a physician to capture a standard length guidewire having a standard diameter (such as 0.036 cm (0.014 in)) within a guide catheter to facilitate exchanging an OTW catheter without the risk of losing the guidewire position or pushing the guidewire distally into a vessel. The captivation catheter includes a captivation portion, such as a balloon, on the distal end that, when inflated, is able to secure a 0.036 cm diameter guidewire within a 90 cm or 100 cm guide catheter. The captivation catheter can be delivered alongside the guidewire and the OTW catheter while both are within the guide catheter and can remain in the guide catheter at all times. The captivation catheter is not delivered over a guidewire, so no guidewire lumen is needed within the captivation catheter. The proximal end of the captivation catheter has a hub with a standard fluid fitting for attachment to a syringe or other inflation device. The distal end of the captivation catheter can have a flexible, atraumatic, radiopaque tip distal to the captivation balloon. The captivation catheter will not contact tissue; it will remain inside the guide catheter and therefore can be characterized by significant pushability without risking vessel damage.

The captivation catheter can be compatible with 6 F to 8 F guide catheters, with a length ranging from about 90 cm to 100 cm (preferably not to exceed about 110 cm). Adequate force to fix a guidewire against the inner lumen of a guide catheter has been determined to be 50 grams of force based on bench testing. In some aspects, the captivation catheter may exert between 200 grams of force and 500 grams of force such that the captivation catheter is compatible with various guidewire and guide catheter combinations. In some aspects, the inflatable portion of the captivation catheter has a nominal inflation pressure of about 12 atmospheres and a rate burst pressure of about 18 atmospheres.

The captivation catheter may be used to stabilize the position of an over-the-wire balloon catheter in an ostial lesion and for the stabilization of a fixed-wire type dilation catheter. Further, the captivation catheter may also be used in conjunction with other therapeutic or diagnostic devices such as atherectomy, laser irradiation, ultrasound, or optical fiber catheters.

Aspects of the present invention include methods of using a captivation catheter. Such methods include the step of intravascularly advancing a guide catheter to an area near an interventional site, such as the ostium of a coronary artery. The guide catheter has a tubular structure that includes a guide catheter lumen having a guide catheter inner surface. The methods can include advancing a guidewire or a treatment catheter for performing an intravascular procedure through the guide catheter lumen. A distal end of the guidewire or the treatment catheter can be advanced past a distal end of the guide catheter and toward a vascular site at which the intravascular procedure is to be performed.

Aspects of the methods of the present invention include advancing a captivation catheter into the guide catheter lumen. The captivation catheter can be used to stabilize the guidewire or the treatment catheter relative to the guide catheter by inflating the captivation balloon located at or near a distal end of the captivation catheter inside the guide catheter lumen. The methods of inflating the captivation balloon can include urging inflation fluid through a helical cut in the elongate member of the captivation catheter and into the captivation balloon. Inflating the captivation balloon urges the guidewire or the treatment catheter against the guide catheter inner surface and fixes the position of the guidewire or the treatment catheter relative to the guide catheter.

Aspects of the methods of the present invention take advantage of the helical cut in the elongate member. For example, advancing the captivation catheter into the guide catheter lumen includes introducing a portion of the elongate member having increased flexibility, relative to other portions of the elongate member, by virtue of the helical cut present on that portion of the elongate member.

Aspects of the methods of the present invention take advantage of the asymmetry in the captivation balloon to expand the captivation balloon in a first direction relative to the elongate member but not a second, opposite direction. The asymmetric expansion of the captivation balloon relative to a longitudinal axis of the captivation catheter can increase the surface area available to fix the position of the guidewire or the treatment catheter.

Aspects of the methods of the present invention include using the treatment catheter, which includes a dilation balloon. Such methods include performing a dilation with the dilation balloon while the captivation catheter secures the position of the treatment catheter relative to the guide catheter. The guide catheter has a guide catheter working length and the method further includes adjusting a working length of the captivation catheter based on the working length of the guide catheter.

According to some aspects of the methods, adjusting the working length of the captivation catheter includes moving a clip between a first position along the captivation catheter to a second position along the captivation catheter to prevent the distal end of the captivation balloon from being positioned distal of the distal end of the guide catheter. Moving the clip between the first and second positions includes depressing portions of the clip, or pinching the clip, adjacent to the arms on the clip. Further, moving the clip between the first and second positions includes viewing a marking or marked regions on the elongate member. The marking or marked regions include a color marking that is visible when the elongate member is at a first orientation in a surgical field but not a second orientation, and an adjacent texture marking, which is visible when the elongate member is at the second orientation in the surgical field but not the first orientation.

According to some aspects of the methods, the guidewire is stabilized relative to the guide catheter by the captivation catheter and the method further includes advancing a treatment catheter through the guide catheter lumen on the guidewire. The guidewire is stabilized relative to the guide catheter by the captivation catheter and the method further includes withdrawing the treatment catheter from the guide catheter. Prior to stabilizing the guidewire relative to the guide catheter by operating the captivation catheter, the method further includes withdrawing a distal end of the treatment catheter to a position proximal of the distal end of the guide catheter.

While the present subject matter has been described in detail with respect to specific aspects thereof, it will be appreciated that those skilled in the art, upon attaining an understanding of the foregoing, may readily produce alterations to, variations of, and equivalents to such aspects. Accordingly, it should be understood that the present disclosure has been presented for purposes poses of example rather than limitation, and does not preclude the inclusion of such modifications, variations, and/or additions to the present subject matter as would be readily apparent to one of ordinary skill in the art.

Table of Reference Numerals

| | |
|---|---|
| 10 | Ostium |
| 12 | Coronary artery |
| 14 | Treatment site |
| 16 | Aorta |
| 100 | Guide catheter |
| 102 | Guide catheter lumen |
| 104 | Guide catheter distal portion |
| 120 | Guidewire |
| 122 | Guidewire distal portion |
| 140 | Treatment catheter |
| 142 | Treatment portion |
| 200 | Captivation catheter |
| 204 | Elongate member |
| 206 | Elongate member outer surface |
| 208 | Elongate member inner surface |
| 210 | Captivation catheter distal portion |
| 212 | Distal wire |
| 215 | Distal wire tip |
| 220 | First marked regions |
| 222 | Second marked regions |
| 230 | Helical cut |
| 250 | Inflatable portion |
| 260 | Inflatable portion proximal section |
| 270 | Strain relief section |
| 280 | Hub |
| 285 | Fluid fitting |
| 300 | Clip |
| 310 | First arm |
| 315 | Groove |
| 320 | Second arm |
| 330 | Grip section |

What is claimed is:

1. A captivation catheter for use with a guide catheter and a guidewire or a treatment catheter, comprising:
an elongate member extending from a proximal hub to a distal end, a distal portion of the elongate member including a helical cut extending through a wall of the elongate member; and
a captivation balloon positioned over the helical cut, the captivation balloon configured for expansion within the guide catheter to secure the guidewire or the treatment catheter between an outer surface of the captivation balloon and an inner surface of the guide catheter,
wherein the helical cut allows for inflation of the captivation balloon and increased shaft flexibility relative to a proximal portion of the elongate member.

2. The captivation catheter of claim 1, wherein a pitch of the helical cut decreases in a proximal-to-distal direction along the distal portion of the elongate member.

3. The captivation catheter of claim 1, wherein a distal portion of the helical cut includes a through hole to inhibit crack propagation.

4. The captivation catheter of claim 1, wherein an outer diameter of the elongate member decreases in a proximal-to-distal direction along the elongate member.

5. The captivation catheter of claim 1, wherein the elongate member includes at least first and second markings indicative of a position of the captivation catheter relative to the guide catheter, the first marking visible when the elongate member is at a first orientation but not a second orientation in a surgical field and the second marking visible when the elongate member is at the second orientation but not the first orientation in the surgical field.

6. The captivation catheter of claim 5, wherein the first marking includes a color marking and the second marking includes a surface texture marking.

7. The captivation catheter of claim 5, wherein the first and second markings are positioned adjacent to each other.

8. The captivation catheter of claim 1, further comprising a clip having an open first end that is attachable to the elongate member and a closed second end that is opposite the first end.

9. The captivation catheter of claim 8, wherein the clip is configured to be removed from the elongate member by depressing portions of the clip adjacent the first end.

10. The captivation catheter of claim 1, wherein a center of the captivation balloon post-expansion is offset relative to an axis of the elongate member.

11. A method, comprising:
intravascularly advancing a guide catheter having a guide catheter lumen defined by a guide catheter inner surface;
advancing a guidewire or a treatment catheter for performing an intravascular procedure through the guide catheter lumen, including advancing a distal end of the guidewire or the treatment catheter past a distal end of the guide catheter and toward a vascular site at which the intravascular procedure is to be performed;
advancing a captivation catheter into the guide catheter lumen; and
stabilizing the guidewire or the treatment catheter relative to the guide catheter by inflating a captivation balloon located at or near a distal end of the captivation catheter inside the guide catheter lumen, including urging inflation fluid through a helical cut in an elongate member and into the captivation balloon so as to urge the guidewire or the treatment catheter against the guide catheter inner surface.

12. The method of claim 11, wherein advancing the captivation catheter into the guide catheter lumen includes introducing a portion of the elongate member having increased flexibility, relative to other portions of the elongate member, by way of the helical cut.

13. The method of claim 11, wherein inflating the captivation balloon includes expanding the captivation balloon in a first direction relative to the elongate member but not a second, opposite direction.

14. The method of claim 11, wherein the treatment catheter includes a dilation balloon and the method further comprises performing a dilation with the dilation balloon while the captivation catheter secures the position of the treatment catheter relative to the guide catheter.

15. The method of claim 11, wherein the guide catheter has a guide catheter working length and the method further comprises adjusting a working length of the captivation catheter based on the working length of the guide catheter.

16. The method of claim 15, wherein adjusting the working length of the captivation catheter includes moving a clip between a first position along the captivation catheter to a second position along the captivation catheter to prevent the captivation balloon from being positioned distal of the distal end of the guide catheter.

17. The method of claim 16, where moving the clip between the first and second positions includes depressing portions of the clip adjacent to a plurality of clip attachment arms.

18. The method of claim 16, wherein moving the clip between the first and second positions includes viewing a marking on the elongate member.

19. The method of claim 18, wherein the marking includes a color marking, which is visible when the elongate member is at a first orientation in a surgical field but not a second orientation, and an adjacent texture marking, which is visible when the elongate member is at the second orientation in the surgical field but not the first orientation.

20. The method of claim 11, wherein the guidewire is stabilized relative to the guide catheter by the captivation catheter and the method further comprises advancing a treatment catheter through the guide catheter lumen and over the guidewire.

21. The method of claim 11, wherein the guidewire is stabilized relative to the guide catheter by the captivation catheter and the method further comprises withdrawing the treatment catheter from the guide catheter.

22. The method of claim 21, wherein, prior to stabilizing the guidewire relative to the guide catheter by operating the captivation catheter, the method further comprises withdrawing a distal end of the treatment catheter to a position proximal of the distal end of the guide catheter.
